(19) United States
(12) Patent Application Publication
(10) Pub. No.: US 2023/0273131 A1
(43) Pub. Date: Aug. 31, 2023

(54) DEVICE FOR TESTING MULTIPLE BIOCHEMICAL INDEXES OF HUMAN BODY AT HOME

(71) Applicant: Lollipop Technology (Hangzhou) Co., Ltd., Hangzhou (CN)

(72) Inventor: WEI ZHANG, Hangzhou (CN)

(21) Appl. No.: 17/660,210

(22) Filed: Apr. 21, 2022

(30) Foreign Application Priority Data
Feb. 25, 2022 (CN) 202210182415.X
Feb. 25, 2022 (CN) 202210182983.X
Feb. 25, 2022 (CN) 202220405968.2

(51) Int. Cl. G01N 21/84; G01N 33/558; A61B 5/00

(52) U.S. Cl. CPC G01N 21/8483; G01N 33/558; A61B 5/0002

(57) ABSTRACT

A device for testing multiple biochemical indexes of the human body at home, includes a test strip and an instrument body. The test strip includes test paper, and the test paper is provided with a sample adding zone, a marker zone, and a reaction zone; the marker zone contains one or more specific binding substances a which are marked by markers and can specifically bind to biochemical substances to be tested, the reaction zone is provided with test lines corresponding to the biochemical substances to be tested, and each test line is at least immobilized with one of a corresponding substance of the same type as the biochemical substance to be tested or a specific binding substance b that can specifically bind to the biochemical substance to be tested; and the instrument body is used with the test strip to identify and analyze the reaction zone on the test paper and output test results. The device can simultaneously test the functions of multiple physiological indexes, and a user can conveniently test multiple physiological indexes at home and combine the multiple biochemical indexes for comprehensive health assessment.

### CROSS-REFERENCE TO RELATED APPLICATIONS

This application claims the benefit and priority of Chinese Patent Application Number 202220405968.2, 202210182983.X, 202210182415.X, which filed on Feb. 25, 2022 with China National Intellectual Property Administration, the disclosures of which are incorporated herein by reference in their entireties.

### BACKGROUND OF THE PRESENT INVENTION

### Field of Invention

The present invention relates to the technical field of physiological test devices, in particular to a device for testing multiple biochemical indexes of the human body at home.

### DESCRIPTION OF RELATED ARTS

With the improvement of living standards, nowadays people pay more and more attention to their own health. For individuals and families, the best way to manage health is to go to the hospital or clinic for regular health examination and make corresponding treatment plans based on the test results. Generally, a considerable proportion of health examination items in the hospital are various biochemical tests on a patient's body fluids such as urine, blood and saliva, and the test results are compared with normal levels to determine possible unhealthy problems of the patient.

Since hospital or clinic examination often requires appointment and queuing and comes with a high price, some test suites emerge now by which patients can collect body fluid samples at home and mail the samples to labs for multiple biochemical tests. Although this method is more convenient to operate, the test period usually takes several days, which is not conducive to timely report the situation on the test day.

There are also some simple household self-test devices, such as thyroid function test kits, but the test devices have the following problems: a test device can only test one physiological index, and cannot test multiple physiological indexes at the same time, so the user cannot combine multiple biochemical indexes for comprehensive health assessment.

To sum up, a test device is required so that a user can conveniently test multiple physiological indexes thereof at home and combine the multiple physiological indexes for comprehensive health assessment, which saves the troubles of appointment and queuing in the hospital or clinic for the user, reduces the frequency of going out, and also reduces the waiting time and expenses of tests.

### SUMMARY OF THE PRESENT INVENTION

To solve the problems that the existing test devices test single items and one test device can only test one physiological index and cannot test multiple physiological indexes at the same time so that a user cannot combine multiple biochemical indexes for comprehensive health assessment, the objective of the present invention is to provide a device for testing multiple biochemical indexes of the human body at home, so that a user can conveniently test multiple physiological indexes thereof at home and combine the multiple physiological indexes for comprehensive health assessment, which saves the troubles of appointment and queuing in the hospital or clinic for the user, reduces the frequency of going out, and also reduces the waiting time and expenses of tests.

The objective of the present invention is achieved by the following technical solutions: a device for testing multiple biochemical indexes of the human body at home, including:

a test strip, wherein the test strip includes test paper, and the test paper is provided with a sample adding zone, a marker zone, and a reaction zone; the marker zone contains one or more specific binding substances a which are marked by markers and can specifically bind to biochemical substances to be tested, the reaction zone is provided with test lines corresponding to the biochemical substances to be tested, and each test line is at least immobilized with one of a corresponding substance of the same type as the biochemical substance to be tested or a specific binding substance b that can specifically bind to the biochemical substance to be tested; the reaction zone is further provided with quality control lines, and each quality control line is quantitatively immobilized with a specific binding substance c capable of binding and capturing the specific binding substance a; and

an instrument body used with the test strip to identify and analyze the reaction zone on the test paper and output test results.

In the present invention, when the test strip is used, a test sample is added to the sample adding zone of the test paper dropwise, the test sample containing at least one biochemical substance to be tested (the biochemical substance to be tested may be luteinizing hormone (LH), follicle-stimulating hormone (FSH), human chorionic gonadotropin (HCG), etc.); the biochemical substance to be tested permeates the marker zone under the chromatographic action of the test paper, and then binds to one corresponding specific binding substance a in the marker zone to form a binding body with a marker, and the binding body continues to move to the reaction zone under the chromatographic action of the test paper; after the binding body reaches the position of a corresponding test line in the reaction zone, since the specific binding substance b immobilized on the test line can specifically bind to the biochemical substance to be tested in the binding body, the binding body will be captured and bound by the specific binding substance b immobilized on the test line; when the concentration of the biochemical substance to be tested in the test sample is higher, more marker is captured and immobilized on the test line; and the content of the marker on the test line is tested and analyzed by the instrument body, to obtain concentration data of the biochemical substance to be tested, so as to obtain a corresponding index. The marker may be a fluorescent marker or colloidal gold or other types of markers. Taking the fluorescent marker as an example, the instrument body can test the concentration by means of fluorescent detection. When the concentration of the antigen to be tested in the test sample is higher, the concentration of the marker immobilized on the corresponding test line is also higher. In this way, when the instrument body performs fluorescent detection, the fluorescent signal on the corresponding test line is stronger, indicating that the concentration is higher.

For a small molecular biochemical substance to be tested such as pregnanediol-3-glucuronide (PDG), a fixed quantity of the corresponding substance of the same type as the biochemical substance to be tested is immobilized on the test line, and the working principle is as follows: when the biochemical substance to be tested is chromatographed to the marker zone, the biochemical substance to be tested will bind to the specific binding substance a marked by the marker in the marker zone to form a binding body (the quantity of the specific binding substance a in the marker zone is fixed and greater than the quantity of the antigen to be tested), the remaining specific binding substance a in the marker zone does not bind to the biochemical substance to be tested and is in a free state, the free specific binding substance a will be chromatographed to the reaction zone together with the binding body, the binding body cannot bind to the corresponding substance on the test line, but the free specific binding substance a marked by the marker will specifically bind to the biochemical substance to be tested on the test line, and the more the biochemical substance to be tested in the test sample (the higher concentration of the biochemical substance to be tested in the sample), the less the marker immobilized on the test line, so the concentration of the biochemical substance to be tested in the test sample also can be reflected by the quantity of the marker on the test line. When the biochemical substance to be tested is an antigenic substance, the specific binding substance a is a specific antibody a that can specifically bind to the antigenic substance, and the specific binding substance b is a specific antibody b that can specifically bind to the antigenic substance.

In the present invention, one or more specific binding substances a for different biochemical substances are provided in the marker zone of the test paper, and specific binding substances b or equivalents for different biochemical substances are immobilized on different test lines in the reaction zone of the test paper, so that the test paper can test a variety of different biochemical substances at the same time, and different test lines on the reaction zone of the test paper can display the concentration information of different biochemical substances, the function of testing multiple physiological indexes at the same time by one test device is realized, and a user can conveniently test multiple physiological indexes thereof at home and combine the multiple physiological indexes for comprehensive health assessment, which saves the troubles of appointment and queuing in the hospital or clinic for the user, reduces the frequency of going out, and also reduces the waiting time and expenses of tests.

In the present invention, quality control lines are provided in the reaction zone of the test paper, and the quality control lines are used to determine whether the current test is valid, so as to ensure the reliability of the test results. The working principle of the quality control line is as follows: the quantity of specific binding substance c immobilized on the quality control line is fixed, so during the test process, the quantitative range of specific binding substance a that can be bound and captured by the specific binding substance c is also definite, that is, the quantitative range of marker that can be immobilized on the quality control line during the test process is also definite, and this range is a valid range; when the instrument body tests the quantity of the marker on the quality control line, if the quantity of the marker on the quality control line is within the valid range, the test data is valid; otherwise, the test result is invalid. Taking the fluorescent marker as an example, the instrument body tests the quantity of the marker on the quality control line by means of fluorescent detection. The stronger the fluorescent signal reflected by the quality control line, the larger the quantity of the marker immobilized on the quality control line; and if the intensity of the fluorescent signal reflected on the quality control line is within a qualified range, the current test is valid, otherwise the current test is invalid.

Preferably, the marker is colloidal gold or a fluorescent marker.

Preferably, the test paper is further provided with an absorption zone. The absorption zone is located on one side of the reaction zone, and the absorption zone is made of a water absorbing material, which is used to absorb the non-captured specific binding substance carrying the marker, so as to ensure the accuracy and reliability of test data.

Preferably, the instrument body includes:

a circuit module;

a light path module, electrically connected to the circuit module, and configured to perform light detection on the reaction zone of the test paper to obtain light detection information, and to send the light detection information to the circuit module, wherein the circuit module processes the light detection information to obtain test results; and

a display screen, electrically connected to the circuit module and configured to display the test results.

After the test sample reacts with the test paper, the reaction zone of the test paper will show different states according to different concentrations of the biochemical substances in the test sample, and then the test strip is put into the instrument body; in some cases, the test strip may be first inserted into the instrument body, and then the sample is added to the test strip; the light path module on the instrument body tests and analyzes the content of the marker on the test line in the reaction zone and obtain light test information, the circuit module processes the information of the light path module and converts the information into test data, and the display screen displays the test data, so that the user can conveniently obtain test results.

Preferably, the light path module includes at least one light source emitter, a light receiver(s) corresponding to the light source emitter(s), and a dichroscope, wherein the dichroscope is used to reflect light emitted by the light source emitter to the reaction zone on the test paper and transmit the reflected light from the reaction zone, and the light receiver is used to receive the reflected light from the reaction zone. The working principle of the light path module is as follows: a light source emitter and a corresponding light receiver constitute a light path detection unit, and each light path detection unit corresponds to a test line or a quality control line on the test reaction zone. The light source emitter emits test light of a specific wavelength, the test light is reflected by the dichroscope and then irradiated on the test line in the reaction zone, the marker on the test line absorbs the test light and emits reflected light of a specific wavelength, and the reflected light is irradiated on the light receiver after passing through the dichroscope. During this process, the larger the quantity of the marker on the test line, the higher the intensity of the reflected light. The light receiver tests the intensity of the reflected light. The intensity of the reflected light can reflect the quantity of the biochemical substance to be tested, then the concentration of the biochemical substance to be tested in the test sample can be calculated, and the corresponding physiological index can be obtained.

Preferably, a filter is provided between the dichroscope and the light receiver.

Preferably, a first condenser is provided between the light source emitter and the dichroscope, and a second condenser is provided between the dichroscope and the reaction zone on the test paper.

Preferably, a chip for storing test item information is provided on the test strip, and an identification module for identifying and reading the chip is provided on the circuit module of the instrument body. Each test strip is provided with a corresponding chip, and the information stored on the chip matches the item information that the test strip can test. When the test strip is put into the instrument body, the identification module on the instrument body can read the information of the chip and give matching test results.

Preferably, a wireless transmission module is provided on the circuit module of the instrument body. The wireless transmission module may be a Bluetooth module or other types of wireless transmission modules, and the wireless transmission module can send the data tested by the instrument body to a device such as a mobile phone.

The beneficial effects of the present invention are: in the present invention, one or more specific binding substances a for different biochemical substances are provided in the marker zone of the test paper, and specific binding substances b or equivalents for different biochemical substances are immobilized on different test lines in the reaction zone of the test paper, so that the test paper can test a variety of different biochemical substances at the same time, and different test lines on the reaction zone of the test paper can display the concentration information of different biochemical substances, the function of testing multiple physiological indexes at the same time by one test device is realized, and a user can conveniently test multiple physiological indexes thereof at home and combine the multiple physiological indexes for comprehensive health assessment, which saves the troubles of appointment and queuing in the hospital or clinic for the user, reduces the frequency of going out, and also reduces the waiting time and expenses of tests.

### BRIEF DESCRIPTION OF THE DRAWINGS

FIG. 1 is a schematic structural illustration of the present invention.

FIG. 2 is an exploded structural illustration of a test strip.

FIG. 3 is a schematic structural illustration of test paper.

FIG. 4 is an exploded structural illustration of an instrument body.

FIG. 5 is a schematic illustration of a light path module.

In the figures: 1. Test strip, 2. Instrument body, 3. Housing, 4. Test paper, 5. Sample adding hole, 6. Protective cover, 7. Display window, 8. Chip, 9. Sample adding zone, 10. Marker zone, 11. Test line, 12. Quality control line, 13. Absorption zone, 14. Shell, 15. Insertion port, 16. Circuit module, 17. Light path module, 18. Display screen, 19. Key, 20. Light source emitter, 21. Light detector, 22. Dichroscope, 23. Filter, 24. First condenser, 25. Second condenser, 26. Reaction zone.

### DETAILED DESCRIPTION OF THE PREFERRED EMBODIMENT

A clear and complete description will be made to the technical solutions in the embodiments of the present invention below with reference to the accompanying drawings in the embodiments of the present invention. Apparently, the embodiments described are only part of the embodiments of the present invention, not all of them. Based on the embodiments of the present invention, all other embodiments obtained by those of ordinary skill in the art fall within the protection scope of the present invention.

Those skilled in the art should understand that, in the disclosure of the present invention, the orientations or positional relationships indicated by the terms “longitudinal”, “lateral”, “upper”, “lower”, “front”, “rear”, “left”, “right”, “vertical”, “horizontal”, “top”, “bottom”, “inner”, “outer”, etc. are based on the orientations or positional relationships shown in the accompanying drawings, and are intended to facilitate the description of the present invention and simplify the description only, rather than indicating or implying that the device or element referred to must have a particular orientation or be constructed and operated in a particular orientation, and therefore, these terms cannot be interpreted as limiting the present invention.

It can be understood that the term “a” should be understood as “at least one” or “one or more”, that is, in one embodiment, the number of an element may be one, and in another embodiment, the number of the element may be more than one, so the term “one” cannot be understood as a restriction on the number.

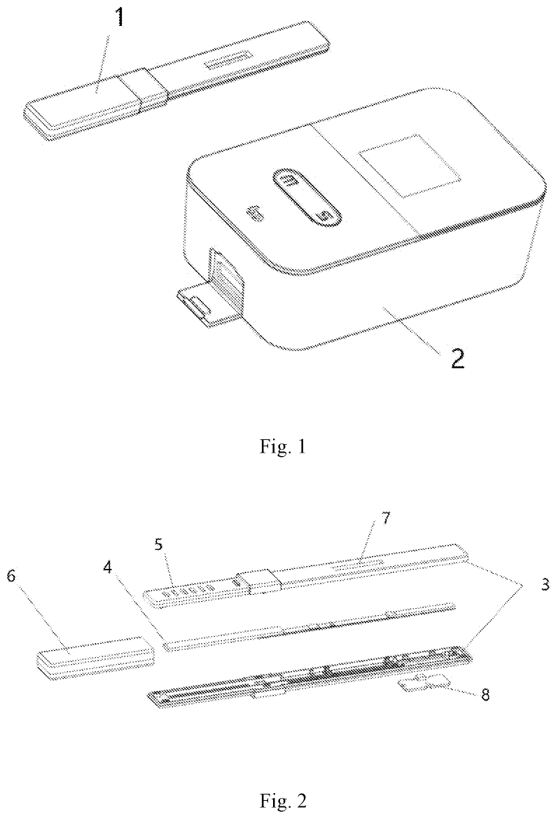

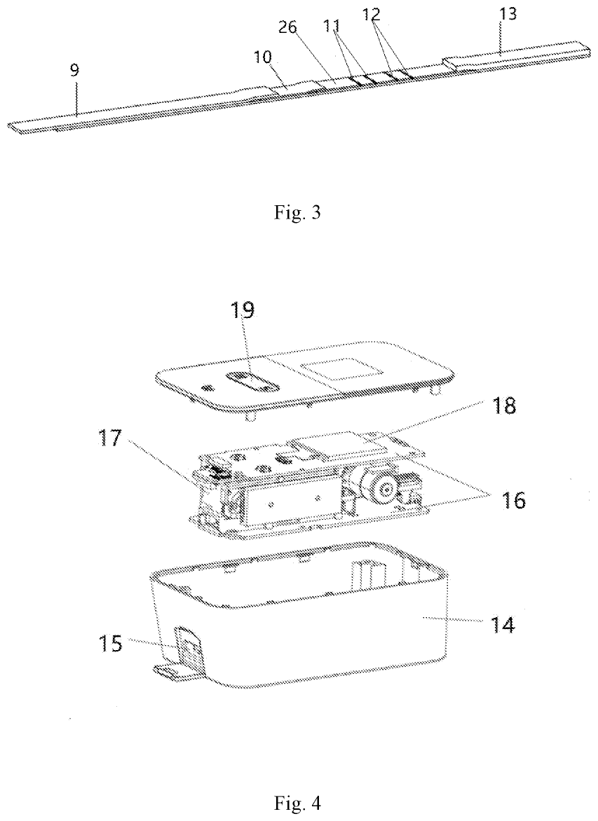

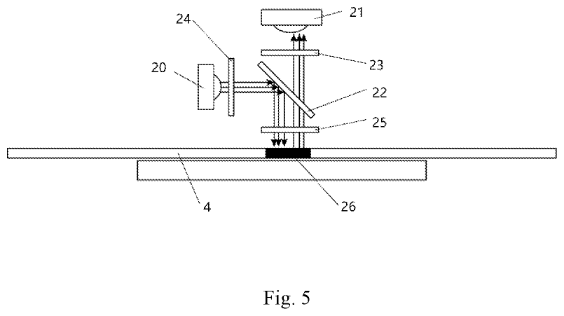

As shown in FIG. 1 to FIG. 5, a device for testing multiple biochemical indexes of the human body at home includes a test strip 1 and an instrument body 2 used with the test strip. The test strip includes a housing 3, test paper 4 arranged in the housing 3, and a protective cover 6. The test paper 4 is provided with a sample adding zone 9, a marker zone 10, a reaction zone 26 and an absorption zone 13 in sequence. The housing 3 is provided with sample adding holes 5 corresponding to the sample adding zone 9, and the sample adding holes 5 are located at one end of the housing 3. Both the sample adding zone 9 and the absorption zone 13 are made of absorbing materials. The housing 3 is provided with a display window 7 corresponding to the reaction zone 26. The protective cover 6 can be sleeved on the end of the housing 3 close to the sample adding holes 5. The marker zone 10 contains one or more specific binding substances a which are marked by markers and can specifically bind to biochemical substances to be tested, the reaction zone is provided with test lines 11 corresponding to the biochemical substances to be tested, and each test line 11 is at least immobilized with one of a corresponding substance of the same type as the biochemical substance to be tested or a specific binding substance b that can specifically bind to the biochemical substance to be tested. The marker may be a fluorescent marker, colloidal gold or other types of markers. The specific binding substance a and the specific binding substance b are determined according to the type of the biochemical substance to be tested. When the biochemical substance to be tested is an antigenic substance, the specific binding substance a is a specific antibody a that can specifically bind to the antigenic substance, and the specific binding substance b is a specific antibody b that can specifically bind to the antigenic substance. The reaction zone is further provided with quality control lines 12, and each quality control line 12 is quantitatively immobilized with a specific binding substance c capable of binding and capturing the specific binding substance a. The housing 3 is further provided with a chip 8, and the chip 8 is configured to store test item information of the test strip.

The instrument body 2 includes a shell 14, a circuit module 16, a light path module 17, a display screen 18, and a key 19. The shell 14 is provided with an insertion port 15 for inserting the test strip. The light path module 17 is electrically connected to the circuit module 16, and is configured to perform light detection on the reaction zone 26 of the test paper 4 to obtain light detection information, and to send the light detection information to the circuit module 16. The circuit module 16 processes the light detection information to obtain test results, and the display screen 18 displays the test results.

The light path module includes at least one light source emitter 20, a light receiver(s) 21 corresponding to the light source emitter(s) 20, and a dichroscope 22. The dichroscope 22 is used to reflect light emitted by the light source emitter to the reaction zone on the test paper and transmit the reflected light from the reaction zone, and the light receiver is used to receive the reflected light from the reaction zone. A filter 23 is provided between the dichroscope 22 and the light receiver 21. A first condenser is provided between the light source emitter and the dichroscope, and a second condenser 25 is provided between the dichroscope 22 and the reaction zone on the test paper.

An identification module for identifying and reading the chip 8 of the test strip 1 is provided on the circuit module 16 of the instrument body 2. After the test strip is put into the instrument body, the identification module on the instrument body can read the information of the chip and provide a matching test result. A wireless transmission module is further provided on the circuit module 16 of the instrument body 2. The wireless transmission module can be a Bluetooth module, the instrument body can be wirelessly connected with a mobile device such as a mobile phone and transmit test data to the mobile device such as the mobile phone, and an APP on the mobile device such as the mobile phone displays the data; meanwhile, the instrument body has a reminding function for setting reminder time according to a user's own situation, and the instrument body can remind the user to timely test after the reminder time.

The working principle of the present invention is as follows: when the test strip is used, a test sample is added to the sample adding zone of the test paper, the test sample contains at least one biochemical substance to be tested (the biochemical substance to be tested may be luteinizing hormone (LH), follicle-stimulating hormone (FSH), human chorionic gonadotropin (HCG), etc.); the biochemical substance to be tested permeates the marker zone under the chromatographic action of the test paper, and then binds to one corresponding specific binding substance a in the marker zone to form a binding body with a marker, and the binding body continues to move to the reaction zone under the chromatographic action of the test paper; after the binding body reaches the position of a corresponding test line in the reaction zone, since the specific binding substance b immobilized on the test line can specifically bind to the biochemical substance to be tested in the binding body, the binding body will be captured and bound by the specific binding substance b immobilized on the test line; when the concentration of the biochemical substance to be tested in the test sample is higher, more marker is captured and immobilized on the test line; and the content of the marker on the test line is tested and analyzed by the light path module on the instrument body to obtain concentration data of the biochemical substance to be tested, so as to obtain a corresponding index. The marker may be a fluorescent marker or colloidal gold or other types of markers. Taking the fluorescent marker as an example, the light path module on the instrument body can test the concentration by means of fluorescent detection. When the concentration of the antigen to be tested in the test sample is higher, the concentration of the fluorescent marker immobilized on the corresponding test line is also higher. In this way, when the light path module performs fluorescent detection, the fluorescent signal on the corresponding test line is stronger, indicating that the concentration is higher.

For a small molecular biochemical substance to be tested such as pregnanediol-3-glucuronide (PDG), a fixed quantity of the corresponding substance of the same type as the biochemical substance to be tested is immobilized on the test line, and the working principle is as follows: when the biochemical substance to be tested is chromatographed to the marker zone, the biochemical substance to be tested will bind to the specific binding substance a marked by the marker in the marker zone to form a binding body (the quantity of the specific binding substance a in the marker zone is fixed and greater than the quantity of antigen to be tested), the remaining specific binding substance a in the marker zone does not bind to the biochemical substance to be tested and is in a free state, the free specific binding substance a will be chromatographed to the reaction zone together with the binding body, the binding body cannot bind to the corresponding substance on the test line, but the free specific binding substance a marked by the marker will specifically bind to the biochemical substance to be tested on the test line, and the more the biochemical substance to be tested in the test sample (the higher concentration of the biochemical substance to be tested in the sample), the less the marker immobilized on the test line, so the concentration of the biochemical substance to be tested in the test sample also can be reflected by the quantity of the marker on the test line. When the biochemical substance to be tested is an antigenic substance, the specific binding substance a is a specific antibody a that can specifically bind to the antigenic substance, and the specific binding substance b is a specific antibody b that can specifically bind to the antigenic substance.

Quality control lines are provided in the reaction zone of the test paper, and the quality control lines are used to determine whether the current test is valid, so as to ensure the reliability of the test results. The working principle of a quality control line is as follows: the quantity of specific binding substance c immobilized on the quality control line is fixed, so during the test process, the quantitative range of specific binding substance a that can be bound and captured by the specific binding substance c is also definite, that is, the quantitative range of marker that can be immobilized on the quality control line during the test process is also definite, and this range is a valid range; when the instrument body tests the quantity of the marker on the quality control line, if the quantity of the marker on the quality control line is within the valid range, the test data is valid; otherwise, the test result is invalid. Taking the fluorescent marker as an example, the instrument body tests the quantity of the marker on the quality control line by means of fluorescent detection. The stronger the fluorescent signal on the quality control line, the larger the quantity of the marker immobilized on the quality control line; and if the intensity of the fluorescent signal reflected on the quality control line is within a qualified range, the current test is valid, and otherwise the current test is invalid.

The working principle of the light path module is as follows: a light source emitter and a corresponding light receiver constitute a light path detection unit, and each light path detection unit corresponds to a test line or a quality control line on the test reaction zone. The light source emitter emits test light of a specific wavelength, the test light is reflected by the dichroscope and then irradiated on the test line in the reaction zone, the marker on the test line absorbs the test light and emits reflected light of a specific wavelength, and the reflected light is irradiated on the light receiver after passing through the dichroscope. During this process, the larger the quantity of the marker on the test line, the higher the intensity of the reflected light. The light receiver tests the intensity of the reflected light. The intensity of the reflected light can reflect the quantity of the biochemical substance to be tested, then the concentration of the biochemical substance to be tested in the test sample can be calculated, and the corresponding physiological index can be obtained.

In the present invention, one or more specific binding substances a for different biochemical substances are provided in the marker zone of the test paper, and specific binding substances b or equivalents for different biochemical substances are immobilized on different test lines in the reaction zone of the test paper, so that the test paper can test a variety of different biochemical substances at the same time, and different test lines on the reaction zone of the test paper can display the concentration information of different biochemical substances, thus realizing the function of testing multiple physiological indexes at the same time by one test device.

The testable biochemical items in the present invention include but are not limited to: LH, FSH, HCG, PDG, progesterone, E1G, ALB, THC, COR, PG I, PG II, FA, PRL, TSH, CRP, 25-OH-D3, SARS-CoV-2/Flu A/Flu B, PSA, NT-proBNP, CCP, HbA1c, AMH, SF, and T.

The present invention can be used for the test and management of various hormones in women's pregnancy preparation period, and its specific method of use is as follows:

In the present application, the first day of a woman's menstruation is defined as the first day of the entire menstrual cycle, which is calculated on the basis of an average women\s menstrual cycle of 28 days. Menstrual days in the following descriptions are calculated on this basis.

1) Fertility test: on day 3 of the menstrual period, a subject's urine is collected as a test sample, the content of follicle-stimulating hormone in the test sample is tested with the testing device, and when the content of follicle-stimulating hormone in the urine is more than 15 mIU/mL, it is considered that the subject's ovarian function has damaged or the subject has entered menopause; at this time, if the subject has a pregnancy preparation plan, the subject should go to the hospital for examination in time after receiving the prompt;

2) Ovulation test: on day 10-12 of the menstrual period, the subject's urine is collected as a test sample, the content of luteinizing hormone in the test sample is tested with the hormone test device, and if the tested content value does not reach 10 mIU/mL or more, the content of luteinizing hormone is tested again at the same time the next day until the tested content value reaches 10 mIU/mL or more; then the content of luteinizing hormone is tested once every 4-6 hours until the tested content value reaches 35 mIU/mL or more, indicating that a ovum will be released within 24-48 hours, intercourse is arranged at this time, and the content of luteinizing hormone in the urine is continuously tested every 4-6 hours until the tested content value is restored to any value below 35 mIU/mL, intercourse is arranged again, and the test is stopped;

3) Ovulation confirmation test: within 7-10 days after ovulation, the subject's urine is collected as a test sample, the content of pregnanediol-3-glucuronide in the test sample is tested with the hormone test device, and if the content values of pregnanediol-3-glucuronide on any two consecutive days within 7-10 days after ovulation exceed 5 μg/mL, it is confirmed that the subject has ovulated;

4) Pregnancy test: on day 10-14 after the ovulation, the content of human chorionic gonadotropin in a urine sample is tested with the hormone test device, and when the HCG content tested on any day of day 10-14 after the ovulation reaches or exceeds 10 mIU/mL, it can be determined that the subject is pregnant.

### Embodiment 2

Embodiment 2 differs from Embodiment 1 in that: in Embodiment 2, the light path module includes a light source emitter 20, a light receiver 21 corresponding to the light source emitter 20, and a dichroscope 22, wherein the dichroscope 22 is used to reflect light emitted by the light source emitter to the reaction zone on the test paper and transmit the reflected light from the reaction zone, and the light receiver is used to receive the reflected light from the reaction zone. A filter 23 is provided between the dichroscope 22 and the light receiver 21. A first condenser is provided between the light source emitter and the dichroscope, and a second condenser 25 is provided between the dichroscope 22 and the reaction zone on the test paper. When the light path module is working, the entire light path module can move along the length of the test paper. During the movement of the entire light path module, the light emitted by the light source emitter 20 is sequentially irradiated to each test line and quality control line on the reaction zone of the test paper, and the reflected light on each test line and quality control line is also received by the light receiver, so that test and analysis of all test lines and quality control lines in the reaction zone can be realized. The remaining features of Embodiment 2 are the same as those of Embodiment 1.

The present invention is not limited to the above-mentioned best embodiments. Any person can derive other products in various forms under the enlightenment of the present invention. However, regardless of any change in shape or structure, all other technical solutions that are the same or similar to the technical solutions of the present application shall fall within the protection scope of the present invention.

1. A device for testing multiple biochemical indexes of the human body at home, comprising:
a test strip (1), wherein the test strip (1) comprises test paper (4), and the test paper (4) is provided with a sample adding zone (9), a marker zone (10), and a reaction zone (26); the marker zone (10) contains one or more specific binding substances a which are marked by markers and can specifically bind to biochemical substances to be tested, the reaction zone is provided with test lines (11) corresponding to the biochemical substances to be tested, and each test line (11) is at least immobilized with one of a corresponding substance of the same type as the biochemical substance to be tested or a specific binding substance b that can specifically bind to the biochemical substance to be tested; the reaction zone (26) is further provided with quality control lines (12), and each quality control line (12) is quantitatively immobilized with a specific binding substance c capable of binding and capturing the specific binding substance a; and
an instrument body (2) used with the test strip to identify and analyze the reaction zone on the test paper (4) and output test results.

2. The device for testing multiple biochemical indexes of the human body at home according to claim 1, wherein the marker is colloidal gold or a fluorescent marker.

3. The device for testing multiple biochemical indexes of the human body at home according to claim 1, wherein the test paper is further provided with an absorption zone (13).

4. The device for testing multiple biochemical indexes of the human body at home according to claim 1, wherein the instrument body comprises:
a circuit module (16);
a light path module (17), electrically connected to the circuit module (16), and configured to perform light detection on the reaction zone (26) of the test paper (4) to obtain light detection information, and to send the light detection information to the circuit module (16), wherein the circuit module processes the light detection information to obtain test results; and
a display screen (18), electrically connected to the circuit module and configured to display the test results.

5. The device for testing multiple biochemical indexes of the human body at home according to claim 4, wherein the light path module (17) comprises at least one light source emitter (20), a light receiver(s) (21) corresponding to the light source emitter(s) (20), and a dichroscope (22), wherein the dichroscope (22) is used to reflect the light emitted by the light source emitter (20) to the reaction zone (26) on the test paper (4) and transmit the reflected light from the reaction zone, and the light receiver (21) is used to receive the reflected light from the reaction zone.

6. The device for testing multiple biochemical indexes of the human body at home according to claim 5, wherein a filter (23) is provided between the dichroscope (22) and the light receiver (21).

7. The device for testing multiple biochemical indexes of the human body at home according to claim 5, wherein a first condenser (24) is provided between the light source emitter (20) and the dichroscope (22), and a second condenser (25) is provided between the dichroscope (22) and the reaction zone (26) on the test paper (4).

8. The device for testing multiple biochemical indexes of the human body at home according to claim 4, wherein a chip (8) for storing test item information is provided on the test strip (1), and an identification module for identifying and reading the chip is provided on the circuit module of the instrument body (2).

9. The device for testing multiple biochemical indexes of the human body at home according to claim 4, wherein a wireless transmission module is provided on the circuit module (16) of the instrument body (2).
